(19) United States
(12) Patent Application Publication
(10) Pub. No.: US 2013/0177547 A1
(43) Pub. Date: Jul. 11, 2013

(54) ANCESTRAL SERINE PROTEASE COAGULATION CASCADE EXERTS A NOVEL FUNCTION IN EARLY IMMUNE DEFENSE

(71) Applicants: Heiko Herwald, Veberod (SE); Ulrich Theopold, Taby (SE); Torsten Loof, Schoeppenstedt (DE); Matthias Mörgelin, Trelleborg (SE); Gerhard Dickneite, Marburg (DE)

(72) Inventors: Heiko Herwald, Veberod (SE); Ulrich Theopold, Taby (SE); Torsten Loof, Schoeppenstedt (DE); Matthias Mörgelin, Trelleborg (SE); Gerhard Dickneite, Marburg (DE)

(73) Assignees: HANSA MEDICAL AB, Lund (SE); CSL BEHRING GMBH, Marburg (DE)

(21) Appl. No.: 13/808,162

(22) PCT Filed: Jul. 20, 2011
(86) PCT No.: PCT/EP2011/062432

§ 371 Date: Mar. 15, 2013

(30) Foreign Application Priority Data
Jul. 22, 2010 (EP) 10170539.0

(51) Int. Cl. A61K 38/45

(52) U.S. Cl. CPC A61K 38/45
USPC 424/94.5; 435/193

(57) ABSTRACT

The present invention relates to blood coagulation factor XIII (FXIII) for treatment and/or prevention of an infection by a microorganism and/or the symptoms associated with said infection, a pharmaceutical composition comprising a pharmaceutically effective amount of said FXIII, a method for the manufacture of a medicament comprising a pharmaceutically effective amount of said FXIII, and a method of treatment comprising administering to a patient in need a pharmaceutically effective amount of said FXIII.

### FIELD OF THE INVENTION

The present invention relates to blood coagulation factor XIII (FXIII) for treatment and/or prevention of an infection by a microorganism and/or the symptoms associated with said infection, a pharmaceutical composition comprising a pharmaceutically effective amount of said FXIII, a method for the manufacture of a medicament comprising a pharmaceutically effective amount of said FXIII, and a method of treatment comprising administering to a patient in need of a pharmaceutically effective amount of said FXIII.

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### BACKGROUND OF THE INVENTION

Serine protease cascades play an important role in many patho-physiologic processes including hemostasis, immune response, and wound healing (for a review, see Page and Di Cera, 2008). Their activation normally occurs by limited proteolysis and coagulation and complement are probably the best-characterized serine proteinase cascades in humans. Phylogenetic studies have shown that the two systems have developed more than 400 million years ago (Davidson et al., 2003; Nonaka and Kimura, 2006) and it has been proposed that they have coevolved from a common ancestral origin in eukaryotes (Krem and Di Cera, 2002). Notably, coagulation and complement cascades share a remarkable degree of convergent evolution with other serine protease cascades regulating for instance Drosophila dorsal-ventral polarity (leading to an activation of Spatzle, the ligand of the Toll receptor) and the horseshoe crab hemolymph clotting system (Krem and Di Cera, 2002). These findings suggest that the basic motifs of some proteolytic cascades existed long before the divergence of protostomes and deuterostomes (Krem and Di Cera, 2001). It should be noted that the latter two systems (activation of Spaetzle and the horseshoe crab hemolymph clotting system) are key components in ancestral immunity, which is to a great deal, if not entirely, dependent on the innate immune system. While the complement system has been considered part of the innate immune system for more than 30 years, it has only been recently appreciated that coagulation also partakes in the early immune defense (for a review, see (Delvaeye and Conway, 2009). In the latter studies, a major focus has been on the clotting cascade's ability to trigger pro- and anti-inflammatory reactions, such as the release of cytokines and activation of protease-activated receptors (PARs). However, little is known as to what extend coagulation can actively contribute to an elimination of an invading microorganism.

In connection with the present invention it was investigated whether the coagulation system exerts antimicrobial activity. Special focus was paid to the role of factor XIII (FXIII), of which the insect homologue (transglutaminase) was recently found to play a protective role in the immune response against bacterial pathogens in a Drosophila infection model (Wang et al., 2010). Streptococcus pyogenes was employed in the present invention, as this bacterium is considered as one of the most important human bacterial pathogens, responsible for at least 18 million cases of severe infections worldwide (1.78 million new cases each year) and more than 500.000 deaths yearly as estimated by the WHO (Carapetis et al., 2005). Infections with S. pyogenes are normally superficial and self-limiting, but they can develop into serious and life-threatening conditions such as necrotizing fasciitis and streptococcal toxic shock syndrome (STSS) which are associated with high morbidity and mortality (for a review, see (Cunningham, 2000). The fact that S. pyogenes can cause local and systemic infections in the same infection model made it an ideal pathogen to be studied in the present invention.

Moreover, many of the hitherto existing antimicrobial agents are rendered ineffective by way of resistances developed against them by a growing number of pathogenic microbial strains. Accordingly, there is a strong demand for new antimicrobial agents, ideally relying on new modes of action which should make it more difficult if not impossible for pathogenic microbes to develop resistance and hence to evade control and eventually killing.

From mechanistic studies using labelled artificial substrates and marker molecules such as biotin-cadaverine (B-cad) during in vitro experiments it is known that the activity of transglutaminase or FXIII as present in normal Drosophila hemolymph or human plasma, respectively, results in the sequestration of the bacteria E. coli or S. aureus in the matrix of a hemolymph or blood clot (Wang et al., 2010).

However, it had not yet been demonstrated that bacteria could be immobilized this way also in vivo. Furthermore, it had been of special interest if a microorganism invading an organism could be prevented from a systemic spreading in the whole body of that host organism by simple immobilization. Finally, it still remained desirable to also eventually achieve killing of the microorganism in the infected host organism.

### BRIEF DESCRIPTION OF THE INVENTION

Surprisingly it has now been found that FXIII causes immobilization of bacteria and generation of antimicrobial activity in the fibrin network of clots in vivo, both in rodents such as mice as well as human tissue.

Surprisingly it has also been found that FXIII, when administered to a living organism infected with Streptococcus pyogenes (S. pyogenes), induced immobilization of these bacteria inside fibrin clots combined with an induction of plasma-derived antimicrobial activity leading to eventual killing by lysis.

Surprisingly, it has also been found that application of human FXIII prevents systemic dissemination of bacteria from the side to infections to organs such as liver and spleen as well into the bloodstream.

Previously this had seemed rather impossible to achieve with this mode of action when attempting to fend off microorganisms which are normally capable of dissolving blood clots by means of their streptokinase (SK) activity or otherwise effecting activation of plasminogen to plasmin and hence fibrinolysis. Indeed, Streptococcus pyogenes is known to carry streptokinase (Kayser, F. H. et al. (1989). Medizinische Mikrobiologie Immunologie, Bakteriologie, Mykologie, Virologie, Parasitologie, 7^{th} edition, Thieme, Stuttgart, 143), which forms a complex with plasminogen. Said complex in turn induces the conversion of plasminogen into plasmin, an endopeptidase which then effects cleavage of fibrin (fibrinolysis) (Pschyrembel Klinisches Wörterbuch (2007). 261^{st} edition, Walter de Gruyter GmbH & Co. KG, Berlin, 602, 1505, and 1846).

In addition, S. pyogenes carries a cell wall attached protein named protein G-related α₂-macroglobulin-binding (GRAB) protein that binds, as its name suggests, to α₂-macroglobulin (α₂-M), which is a human protease inhibitor. It has been suggested that the binding of α₂-M by GRAB and thus to the bacterial surface facilitates bacterial infection by S. pyogenes (a group A streptococcus or GAS) in two ways: removal of α₂-M reduces its inhibitory activity, thereby maintaining a certain level of activity of proteases for a more efficient spreading of bacteria through the tissue of the invaded host, while the bacterium itself remains protected against proteases, for it now carries the inhibitors against them directly on its surface (Toppel et al., 2003). It should be noted, however, that there is a third aspect to this: α₂-M being a protease inhibitor also regulates fibrinolysis in that it acts as an inhibitor to the step of activation of plasminogen to plasmin (Pschyrembel Klinisches Wörterbuch (2007). 261^{st} edition, Walter de Gruyter GmbH & Co. KG, Berlin, 602). When a bacterial surface protein such as GRAB binds to α₂-M and therewith lowers its plasma concentration, the inhibitory activity of α₂-M on plasminogen activation is likewise reduced so that fibrinolysis will be increased.

Accordingly, in principle S. pyogenes is capable of interfering with the fibrinolysis control mechanism in two ways, i.e. enhancing fibrinolysis directly by plasminogen activation and indirectly by reducing plasminogen inhibition. Therefore, S. pyogenes had been expected to rather evade entrapment within fibrin clots and thus immobilization.

The present invention thus provides
(1) blood coagulation factor XIII (FXIII) for treatment and/or prevention of an infection by a microorganism and/or the symptoms associated with said infection;
(2) a pharmaceutical composition comprising a pharmaceutically effective amount of the FXIII as defined under (1) and one or more substances selected from the group consisting of human albumin, glucose, sodium chloride, water and HCl or NaOH for adjusting the pH for treatment and/or prevention of an infection by a microorganism and/or the symptoms associated with said infection;
(3) a method for the manufacture of a medicament comprising a pharmaceutically effective amount of the FXIII or the pharmaceutical composition as defined under (2) for treatment and/or prevention of an infection by a microorganism and/or the symptoms associated with said infection; and
(4) a method of treatment comprising administering to a patient in need a pharmaceutically effective amount of the FXIII or of the pharmaceutical composition as defined under (2) for treatment and/or prevention of an infection by a microorganism and/or the symptoms associated with said infection.

### DETAILED DESCRIPTION OF THE INVENTION

Phylogenetically conserved serine protease cascades play an important role in invertebrate and vertebrate immunity. The mammalian coagulation system can be traced back some 400 million years and it shares homology with ancestral serine proteinases cascades involved for instance in Toll receptor signaling in insects and release of antimicrobial peptides during hemolymph clotting. The present invention shows that bacteria-evoked induction of coagulation leads to an immobilization of microorganisms inside the clot and the generation of antimicrobial activity. Thus, an ancestral serine protease coagulation cascade exerts a novel function in early immune defense. The entrapment is mediated via crosslinking bacteria to fibrin fibers by the action of factor XIII (FXIII), an evolutionarily conserved transglutaminase. Infected FXIII^{-/-} mice show severe signs of pathologic inflammation and treatment of wildtype animals with FXIII dampens bacterial dissemination. Bacterial killing and crosslinking to fibrin networks was also monitored in tissue biopsies from patients with streptococcal necrotizing fasciitis supporting the concept that coagulation is part of the early innate immune system.

The present invention thus demonstrates that
Induction of coagulation exerts bacterial immobilization and antimicrobial activity
Factor XIII^{-/-} mice develop more severe infections than wildtype animals
Bacterial entrapment and killing are recorded in biopsies of infected patients, and
Treatment with FXIII prevents bacterial dissemination in infected mice.

Sensing inflammation and a fast elimination of an invading microorganism are key features of the early immune response to infection. In particular, potential ports of microbial entry are at great risk and they therefore need special protection. Thus, the immune system has developed mechanisms that allow an efficient clearance of for instance inhaled (for example with the help of mannose-binding lectin (Eisen, 2010)) or swallowed (for example by the action of intestinal mucins (Dharmani et al., 2009)) pathogens. Wounds present another port of entry and they bear a great risk to promote infections that allow microorganisms to enter a circulatory system.

To prevent their dissemination and eventual systemic complications, it is of great importance that the host's defense system is activated as soon as wound sealing begins. It therefore appears likely that coagulation plays an important role in these very early processes. However, the extent and underlying mechanisms of this contribution to immunity are little understood.

Here it is shown for the first time that, in addition to its proinflammatory role, coagulation plays an active role in the containment and elimination of bacterial infections. The data obtained for the present invention support a model based on two separate mechanisms, involving a FXIII-triggered covalent immobilization of microorganisms inside the fibrin network and the generation of antimicrobial activity. It was found that clotting is activated at the bacterial surface via the intrinsic pathway of coagulation also referred to as the contact system or kallikrein/kinin system. Apart from bacteria (for a review see (Frick et al., 2007)), also fungi (Rapala-Kozik et al., 2008) and viruses (Gershom et al., 2010) have been reported to interact with the contact system, supporting the notion that contact activation is subjected to the principles of pattern recognition (Opal and Esmon, 2003). Notably, the system is activated within seconds and leads to the release of antimicrobial peptides (Frick et al., 2006; Nordahl et al., 2005) and inflammatory mediators (for a review see (Leeb-Lundberg et al., 2005)) further supporting its role in early innate immunity. In addition to generation of antimicrobial peptides due to activation of the intrinsic pathway of coagulation, also processing of thrombin has recently been shown to release host defense peptides with a broad specificity (Papareddy et al., 2010). However, the extent to which theses peptides contribute to the antimicrobial activity seen in the present invention needs to be clarified.

The in vivo data presented with this invention show that the lack of FXIII evokes pathologic inflammatory reactions illustrated by a massive neutrophil influx to the site of infection and subsequent tissue destruction as seen in the infected mice. The inability to immobilize bacteria leads to a dramatic increase of the intrinsic-driven clotting time in these animals, which is a sign that the infection became more systemic in the knock-out than in wildtype mice. Human plasma FXIII is fully active in mice (Lauer et al., 2002) and as a proof of concept the human protein was administered in a murine infection model. When wildtype mice were treated with human plasma FXIII, it was recorded that bacterial dissemination was significantly reduced compared to non-treated mice. These results underline the importance of FXIII in the early defense against an invading pathogen and they suggest that FXIII is an interesting target for the development of novel antimicrobial therapies.

Clotting has been previously implicated in immunity in invertebrate models, where its immune function is more visible due to the lack of redundancy with adaptive effector mechanisms. One of the best studied examples is the clotting system of horseshoe crabs, which is triggered by minute amounts of bacterial elicitors, such as LPS, leads to the production of antimicrobial activity and communicates with other effector systems. In a similar way there may be cross-talk between complement and blood clotting for example via the binding of ficolin to fibrin/fibrinogen (Endo et al., 2009). The picture that emerges from evolutionary comparisons is that proteolytic cascades and their constituent proteases are used as flexible modules, which can be triggered by endogenous as well as exogenous microbial elicitors (Bidla et al., 2009). Even one and the same proteolytic event can be activated by distinct elicitors in different contexts. One such example is the cleavage of the Drosophila protein Spaetzle, which may act as a key signal both during development and in the immune system. In both cases cleaved Spaetzle binds to Toll, the founding member of the TLR family. In a similar way it is shown here that blood clotting, which so far has been studied mostly in the context of its physiological hemostatic function, plays a key role in immunity both as an effector mechanism and by communicating with other branches of the immune system. This leads to a fast and efficient instant immune protection, which keeps infections localized and leaves additional time for other effector mechanisms to be activated.

As used by the present invention, factor XIII or blood coagulation factor XIII (FXIII) is a plasma transglutaminase that stabilizes fibrin clots in the final stages of blood coagulation. Thrombin-activated FXIII catalyzes formation of covalent crosslinks between gamma-glutamyl and epsilon-lysyl residues of adjacent fibrin monomers to yield the mature clot. FXIII circulates in plasma as a heterotetramer composed of 2 A-subunits and 2 B-subunits. The A-subunit contains the active site of the enzyme and is synthesized by hepatocytes, monocytes, and megakaryocytes. The B-subunit serves as a carrier for the catalytic A-subunit in plasma and is synthesized by the liver.

The FXIII A-subunit gene belongs to the transglutaminase family, which comprises at least 8 tissue transglutaminases. These enzymes crosslink various proteins and are involved in many physiological and pathological processes, such as hemostasis, wound healing, tumor growth, skin formation, and apoptosis. Similar to tissue transglutaminases, FXIII participates in tissue remodelling and wound healing, as can be inferred from a defect in wound repair observed in patients with inherited FXIII deficiency. FXIII also participates in implantation of the embryo during normal pregnancy; women homozygous for FXIII deficiency experience recurrent miscarriages.

One source of FXIII according to the present invention is FXIII concentrate, e.g. Fibrogammin® P250/1250 (CSL Behring). A concentrate of FXIII can be lyophilised FXIII, e.g. a powder or a FXIII lyophilisate dissolved in water.

The FXIII is usually isolated from human blood plasma, but can also be provided as a recombinant protein using recombinant DNA techniques as known in the art. In general the FXIII according to the invention can either manufactured from plasma, placenta or by methods of genetic engineering (recombinant or transgenic).

In contrast to mere FXIII replacement therapies the objective of which is to achieve normal, healthy plasma levels of FXIII for individuals suffering from a congenital or acquired deficiency of FXIII, FXIII is employed according to the present invention in order to treat and/or prevent an infection by a microorganism, i.e. as kind of an antibiotic or inducer of antibiotic activity. In doing so FXIII is administered to a patient so that the FXIII concentration in the blood plasma of that patient is increased above the FXIII concentration in the blood plasma of a healthy individual, i.e. FXIII can be administered to a patient who does not suffer from a congenital or acquired FXIII deficiency. However, FXIII according to the present invention might be administered for both reasons or indications, i.e. in order to treat a congenital or acquired deficiency of FXIII and at the same time for treating and/or preventing a microbial infection; in such situations the overall dose of FXIII administered has to be higher than in case of only a single indication.

The FXIII can be administered to a patient systemically or topically, preferred is a topical administration at the site of infection if this site can be identified so that spreading from the site of infection is more effectively and rapidly controlled and/or fully prevented. Administration is generally effected by injection, in case of a systemic application an intravenous injection is generally preferred. Other routes of administration the FXIII can be interarterial, subcutaneous, intramuscular, intradermal, inraperitoneal, intracutaneous, inralumbal or intrathecal.

Typical dosage regimens for administration of FXIII according to the present invention require the administration of 5 to 1000 international units (IU) of FXIII per kg body weight, preferably 5 to 500 IU of FXIII per kg body weight, more preferably 5 to 300 IU of FXIII per kg body weight, yet more preferably 5 to 250 IU of FXIII per kg body weight, still more preferably 10 to 200 IU of FXIII per kg body weight. Preferably the FXIII is administered once or up to three times per day.

The FXIII administration has effects such as dampening systemic dissemination, immobilization and/or killing of the microorganism in the body of a patient.

The microorganisms targeted by the present invention can be of relatively high virulence in that they are capable of supporting or enhancing fibrinolysis, capable of activating plasminogen, and/or have plasminogen activating proteins selected from the group consisting of streptokinase (beta-hemolytic streptococci), staphylokinase (Staphylococcus aureus), protein Pla (Yersinia pestis), fibrinolytic enzymes, compounds that activate fibrinolysis or other bacterial proteins for instances from the species Borrelia burgdorferi, Escherichia coli, Fusobacterium nucleatum, Helicobacter pylori, Mycoplasma fermentans, Neisseria gonorrhoeae, Neisseria meningitidis, Pseudomonas aeruginosa, Salmonella enteritidis, Salmonella typhimurium.

Additionally or alternatively, said microorganisms are capable of supporting or enhancing fibrinolysis by carrying at least one surface and/or cell wall protein capable of lowering the plasma concentration of at least one inhibitor of plasminogen activation, said protein being preferably selected from the group consisting of protein GRAB (Streptococcus pyogenes), aureolysin (Staphylococcus aureus), secreted neutral metalloproteases of Bacillus anthracis, and secreted proteases of Peptostreptococcus micros.

The microorganisms according to the present invention can be selected from the group consisting of bacteria, yeasts, viruses and multicellular parasites. Preferably the microorganism is a bacterium having a solid cell wall and/or being Gram-positive, more preferably a bacterium of the family of aerobe and facultative anaerobe coccobacilli, yet more preferably a Streptococcaceae, still more preferably a beta-hemolytic Streptococci, most preferably the microorganism is Streptococcus pyogenes.

The type of infection can be selected from one or more tissue of the group consisting of skin, respiratory system, throat, lung, spleen, liver, kidney, cardiovascular system, heart, central nervous system, digestive system, genitourinary system, muscles and soft tissues. However, a systemic infection of the body of the patient can also be successfully treated.

Symptoms associated with an infection are typical symptoms accompanying infections by the microorganisms targeted by the present invention such as inflammation, headaches, fever, diarrhea, pain, loss of consciousness or a combination of one or more of them.

A pharmaceutical composition comprises a pharmaceutically effective amount of the FXIII of the present invention and one or more substances selected from the group consisting of human albumin, glucose, sodium chloride, water and HCl or NaOH for adjusting the pH. The preferred pH of the pharmaceutical composition is between 7.0 and 7.6, more preferably between 7.2 and 7.4. The pharmaceutical composition can further comprise pharmaceutical carriers, excipients and aids as generally known in the art.

A pharmaceutical effective amount according to the present invention is an amount of FXIII, its concentrate or the pharmaceutical composition which ensures an initial concentration of FXIII in the patient's blood plasma of up to 10 fold at its normal level, preferably up to 5 fold at its normal level. On the other site the initial concentration of FXIII in the patient's blood plasma is at least 250% of the normal FXIII activity.

### DESCRIPTION OF THE FIGURES

### Meaning of Certain Abbreviations

CFU: colony forming unit(s)
OD: optical density
OD405: optical density measured at a wavelength of 405 nm

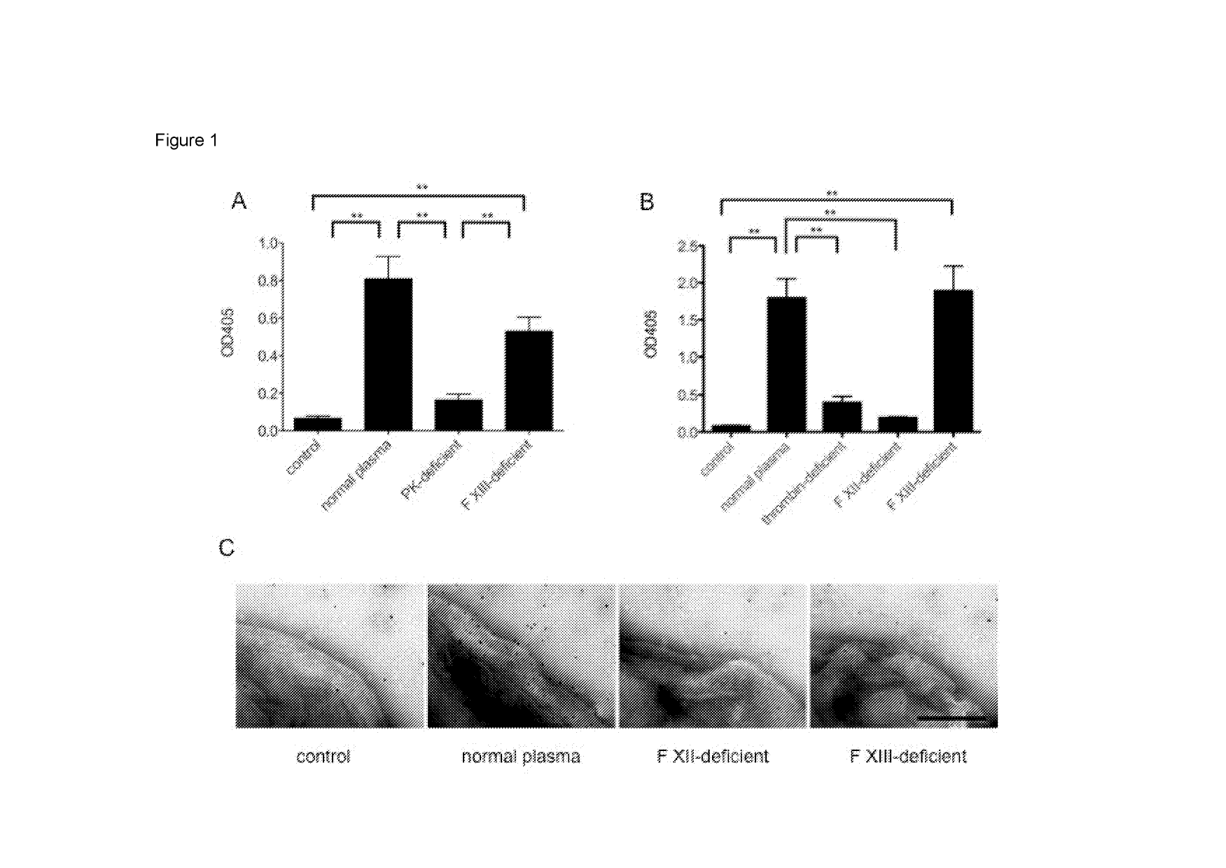

FIG. 1: Activation of the contact system and FXIII on the bacterial surface

(A) AP1 bacteria in Tris containing 50 μM ZnCl₂ were incubated with human normal, PK-deficient, or FXIII-deficient plasma for 30 min. Bacteria were then washed and resuspended in a substrate solution for the measurement of the plasma kallikrein activity on the surface of S. pyogenes.

(B) S. pyogenes in Tris containing 50 μM ZnCl₂ were incubated with normal, thrombin-, F XII-, and FXIII-deficient plasma in the presence of CaCl₂ and phospholipids for 30 min. Bacteria were washed and resuspended in a substrate solution to measure the thrombin activity. Both figures represent the mean±SD of three independent experiments.

(C) AP1 bacteria were incubated in sodium citrate alone, normal plasma, F XII-, or FXIII-deficient (plasma diluted 1/100 in sodium citrate) in the presence of ZnCl₂, CaCl₂, phospholipids, and the gold-labeled antibody against N-epsilon-gamma-glutamyl-lysine for 15 min and afterwards analyzed by negative staining electron microscopy. The scale bar represents 100 nm.

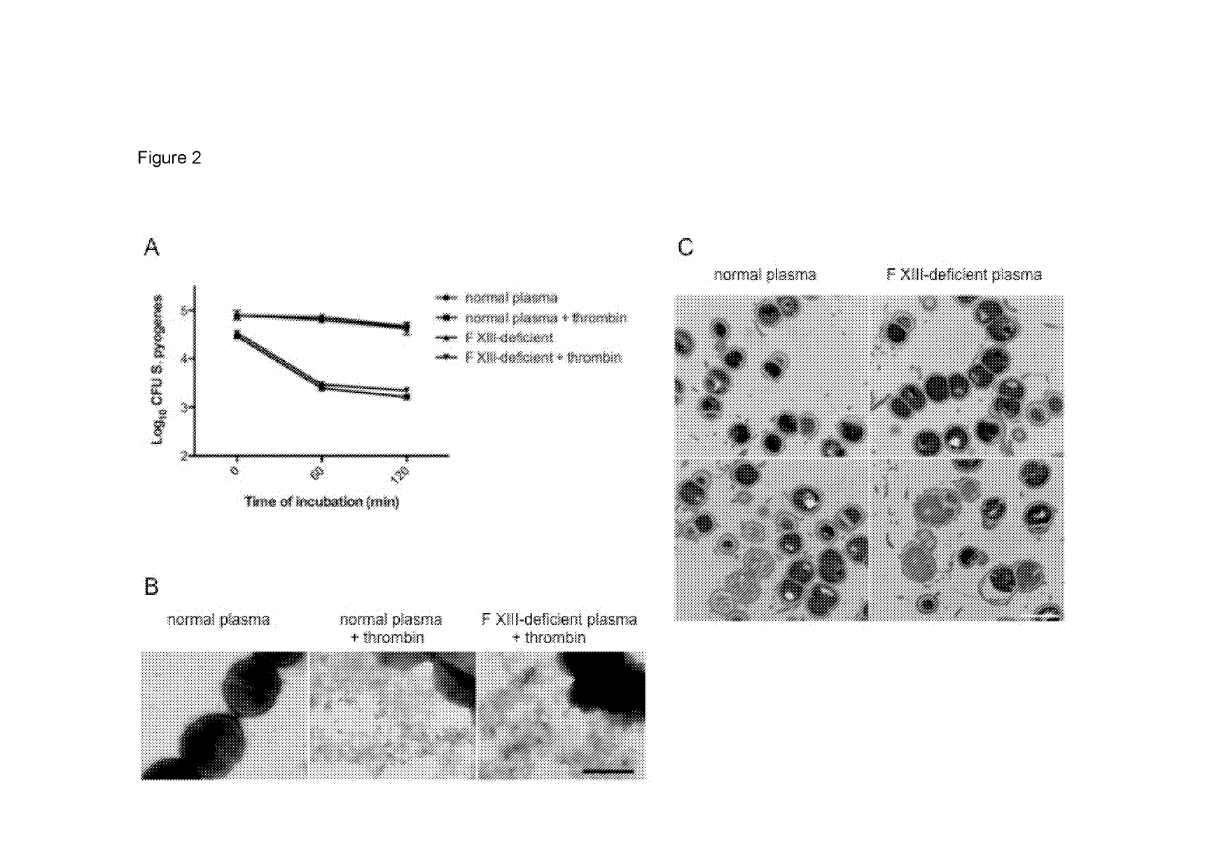

FIG. 2: Thrombin-activated plasma displaces antimicrobial activity

(A) AP1 bacteria were incubated with thrombin-activated normal plasma or FXIII-deficient plasma (1/100 diluted). After indicated time points bacterial numbers were determined by plating of serial dilutions onto blood agar. Bacteria incubated with non-activated normal plasma or FXIII-deficient plasma served as controls. The figure represents the mean±SD of three independent experiments.

(B) AP1 bacteria were incubated in normal plasma (left panel), thrombin-activated normal plasma (middle panel), or thrombin-activated FXIII-deficient plasma (right panel) as described in Experimental Procedures and subjected to analysis by negative staining electron microcopy. The scale bar represents 1 μm.

(C) AP1 bacteria were incubated with normal or FXIII-deficient plasma and clotting was initiated by the addition of thrombin. Thin sectioned clots before (upper lane) and after 1 h at 37° C. (lower lane) are shown. Similar amounts of dead bacteria were detected in both samples after incubation. The scale bar indicates 1 μm.

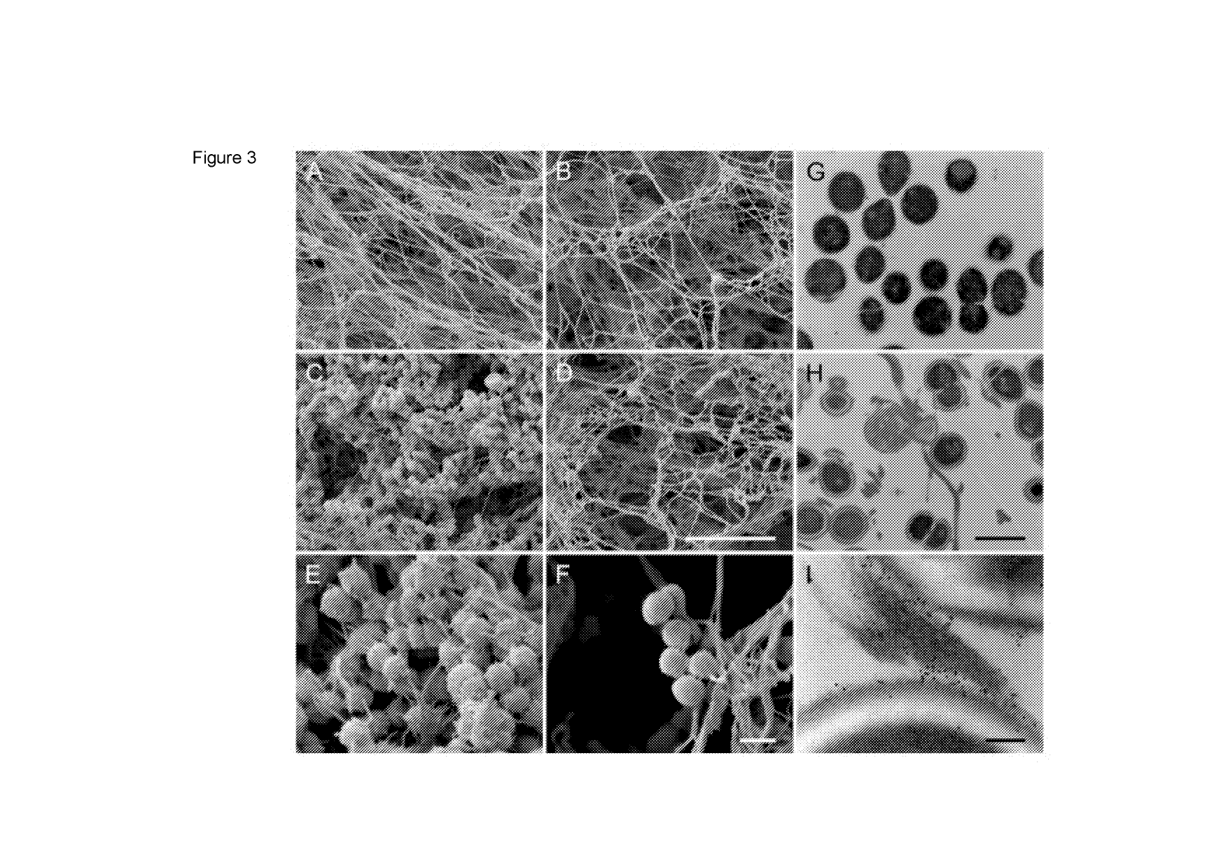

FIG. 3: Entrapment and immobilization of S. pyogenes inside the clot

Scanning electron micrographs showing the structure of clots generated from normal plasma (A, C, E) or FXIII-deficient plasma (B, D, F) in the absence (A, B) or presence (C-F) of bacteria. The scale bars represent 10 μm in A-D and 1 μm in E-F, respectively. The transmission electron micrographs depict S. pyogenes alone (G), after exposure to thrombin-activated plasma (H), and after exposure to plasma followed by immunostaining with a gold-labeled N-epsilon-gamma-glutamyl-lysine antibody recognizing the FXIII crosslinking site (J). Scale bars correspond to 1 μm in G and H, and to 100 nm in J, respectively.

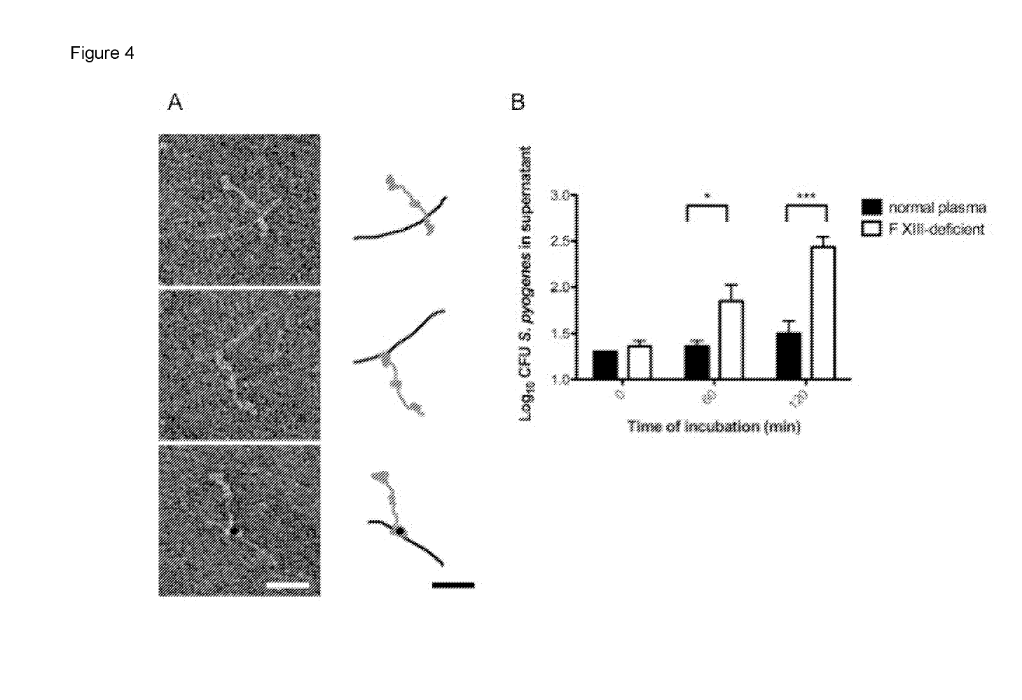

FIG. 4: FXIII crosslinks the streptococcal M1 protein with fibrinogen leading to immobilization of bacteria within the clot

(A) The electron micrographs show negatively stained human fibrinogen (characterized by three domains) in complex with rM1-protein (elongated) before (upper panel) and after FXIII crosslinking (middle panel). Crosslinking was detected by immunostaining the fibrinogen M1 protein complex with the gold-labeled antibody against N-epsilon-gamma-glutamyl-lysine (lower panel). A schematic drawing of the fibrinogen (grey) and M1 protein (black) is included to highlight the interaction between fibrinogen and M1 protein. The scale bars represent 25 nm.

(B) Bacteria were incubated with normal or FXIII-deficient plasma and clotting was initiated by the addition of thrombin. Clots were washed briefly, covered with THB-medium and further incubated at 37° C. After indicated time points bacterial numbers were determined by plating of serial dilutions of the supernatant onto blood agar. The figure represents the mean±SD of three independent experiments.

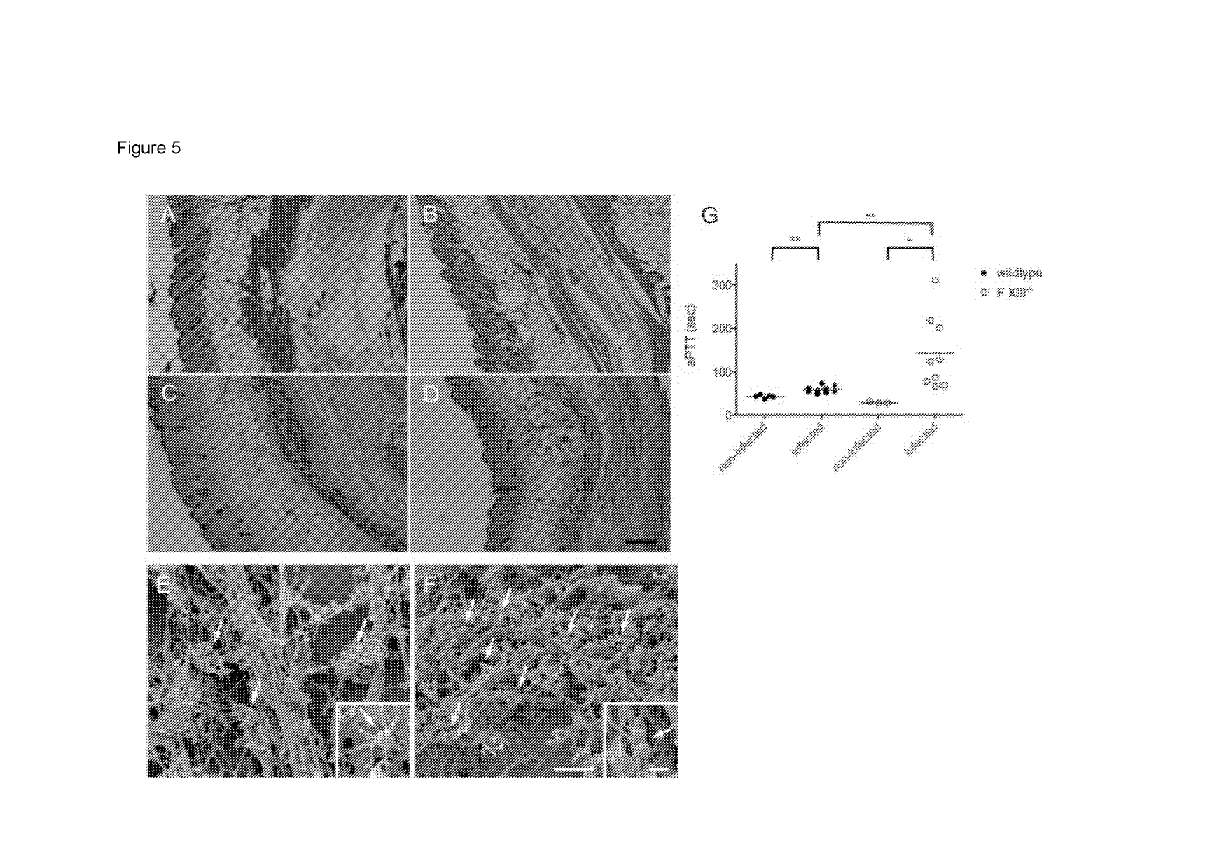

FIG. 5: Subcutaneous infection of wildtype and FXIII^{-/- mice with} S. pyogenes

Haematoxilin/eosin stained representative tissue sections from non-infected (A, B) and infected (24 h; C, D) wildtype (A, C) and FXIII^{-/- (B, D) mice are shown. The scale bar represents} 500 μm.

Scanning electron micrographs depict biopsies from wildtype (E) and FXIII^{-/- (F) mice. Scale bars correspond to} 10 μm and to 1 μm in the inserts.

(G) Activated partial thromboplastin time (aPTT) measured in plasma from non-infected and infected wildtype and FXIII^{-/- mice (}24 h after infection). Data are presented as mean±SD value of plasma samples obtained from 3 or 5 non-infected and 9 infected animals obtained from three independent experiments.

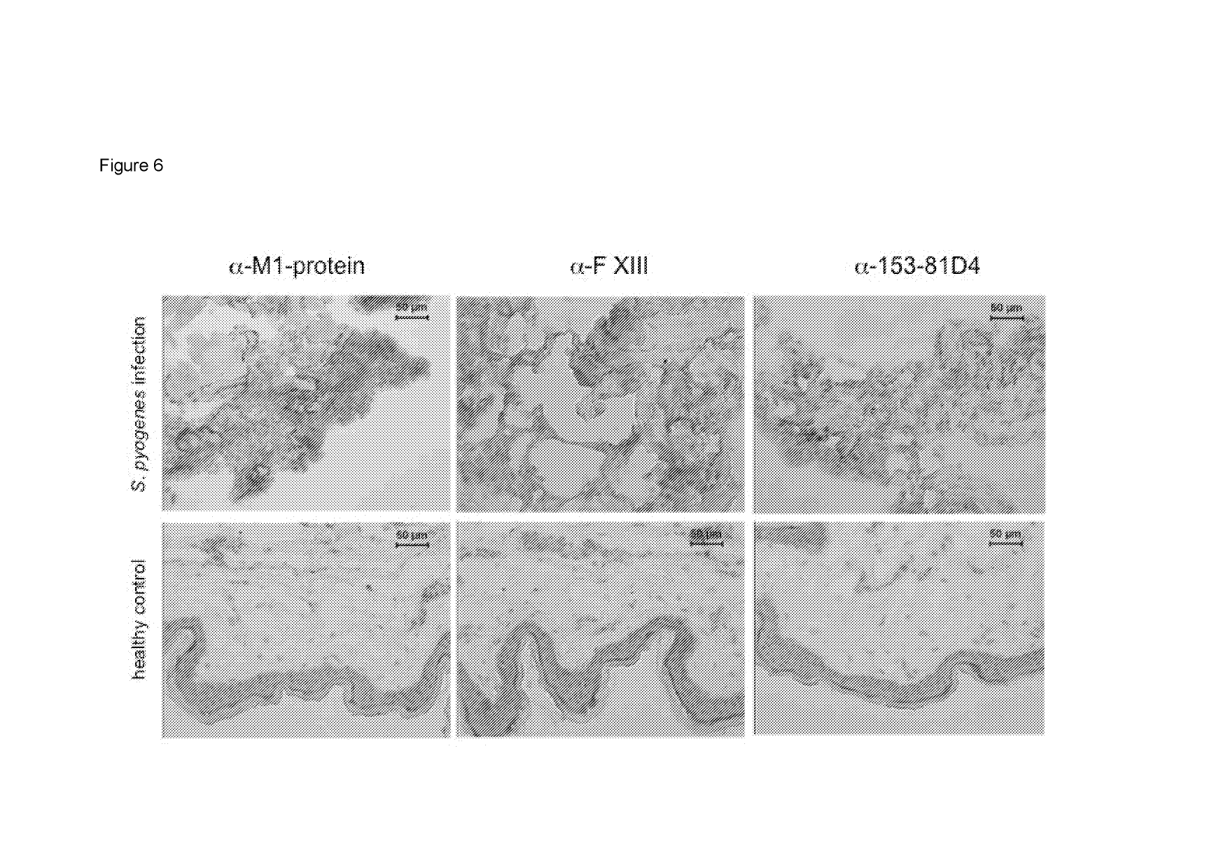

FIG. 6: Immunohistochemical analysis of human biopsies

Tissue biopsies were obtained from patients with necrotizing fasciitis caused by S. pyogenes (upper panel) and healthy volunteers (lower panel). The biopsies were sectioned and immunohistochemically stained for streptococcal M1-protein, FXIII, and N-epsilon-gamma-glutamyl-lysine. Stainings without primary antibodies were negative (data not shown). The scale bar correspond to 50 μm.

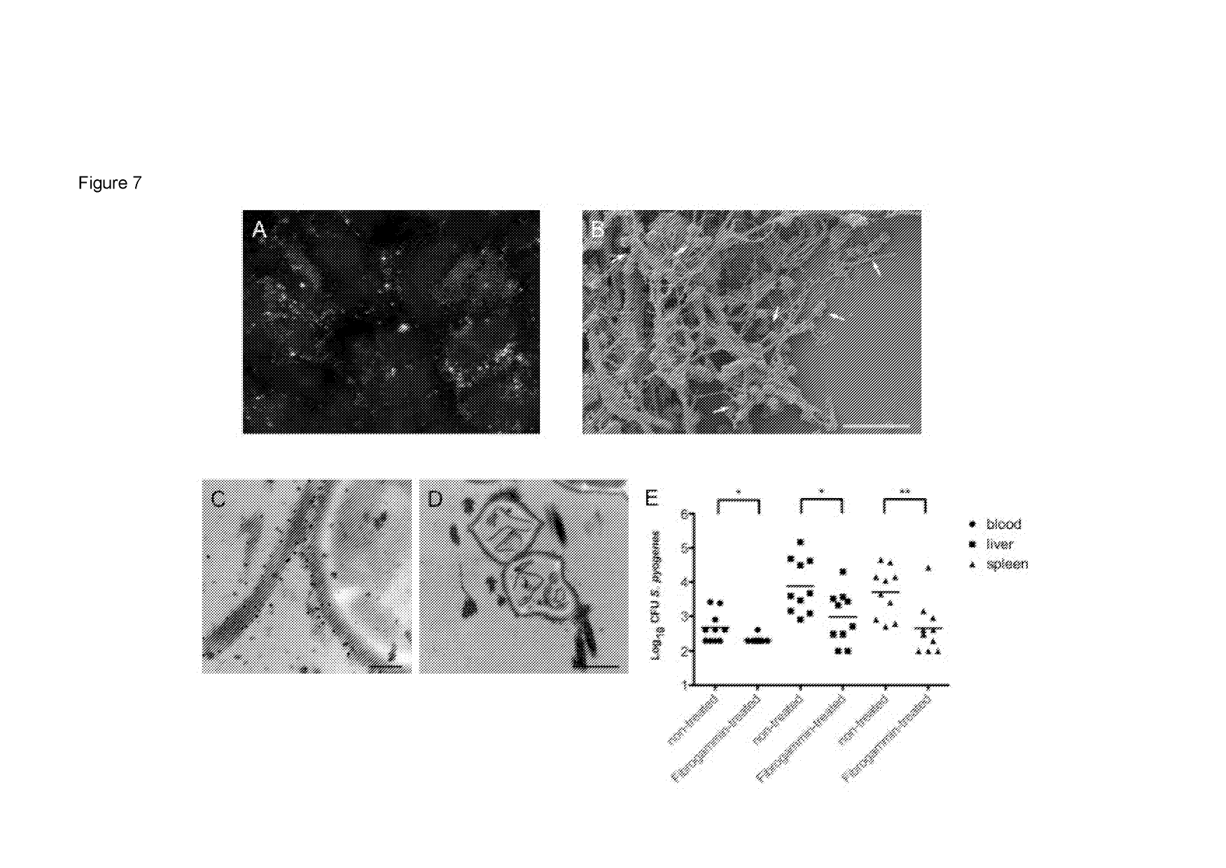

FIG. 7: Co-localization of M1 protein and FXIII crosslinking and bacterial dissemination in FXIII treated mice

(A) Tissue biopsies from patients with streptococcal necrotizing fasciitis were sectioned and immunofluorescently stained for M1 protein (green) in combination with anti N-epsilon-gamma-glutamyl-lysine (red). Cell nuclei are stained in blue with DAPI. Bar indicates 10 μm.

(B) Scanning electron microscopy showing bacteria entrapped in the fibrin network in a biopsy from a patient with streptococcal necrotizing fasciitis. Scale bar indicates 5 μm.

(C) Transmission electron micrograph displaying FXIII-mediated crosslinking of bacterial surface proteins to the fibrin network by detection of the gold-labeled antibody against N-epsilon-gamma-glutamyl-lysine. The scale bar represents 100 nm.

(D) Transmission electron microscopy shows dead bacteria inside a fibrin clot in a biopsy from a patient with streptococcal necrotizing fasciitis. The scale bar represents 0.5 μm.

(E) Mice received a subcutaneous injection of S. pyogenes and were treated with Fibrogammin®P 3 h after infection. Non-treated mice served a control. 24 h after infection mice were sacrificed and bacterial load in blood, liver, and spleen was determined. Data are presented as mean of 10 mice per group and obtained from three independent experiments.

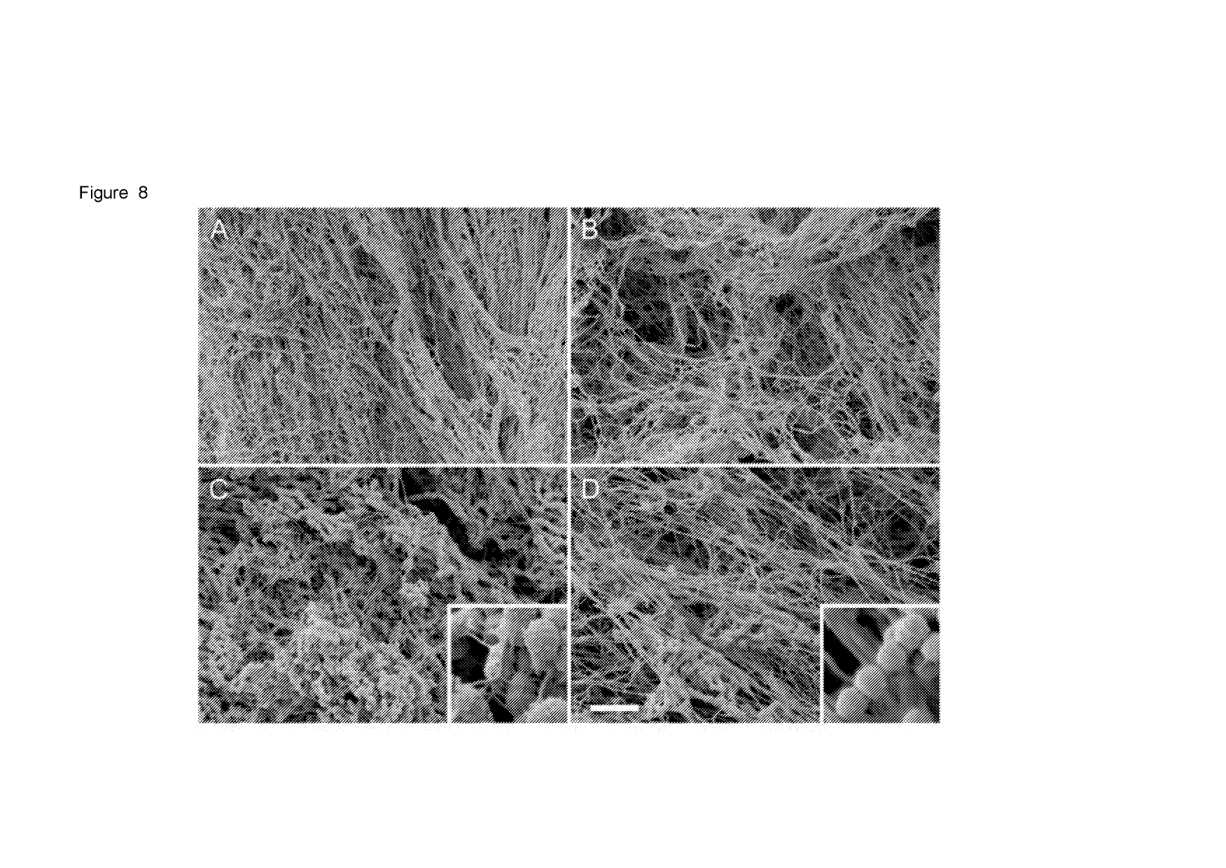

FIG. 8: FXIII-dependent entrapment of S. pyogenes KTL3 in clots generated from murine plasma

Scanning electron micrograph displaying FXIII-dependent entrapment of S. pyogenes in clots generated from murine plasma. Plasma obtained from wildtype (A, C) and FXIII^{-/- mice (B, D) was incubated in the absence and presence of} 2×10⁹ CFU of S. pyogenes strain KTL3 and clotting was initiated by the addition of thrombin. Similar to the results with human plasma, large amounts of S. pyogenes are captured within the clot generated from wildtype (normal) plasma (C) whereas only some few bacteria are found in the FXIII-deficient clot (D). A closer view on the bacteria revealed strong interactions of the surface of S. pyogenes with the fibrin network in the wildtype, but not in the clot lacking FXIII (insets in C and D). The scale bar represents 10 μm respectively 1 μm in the inserts.

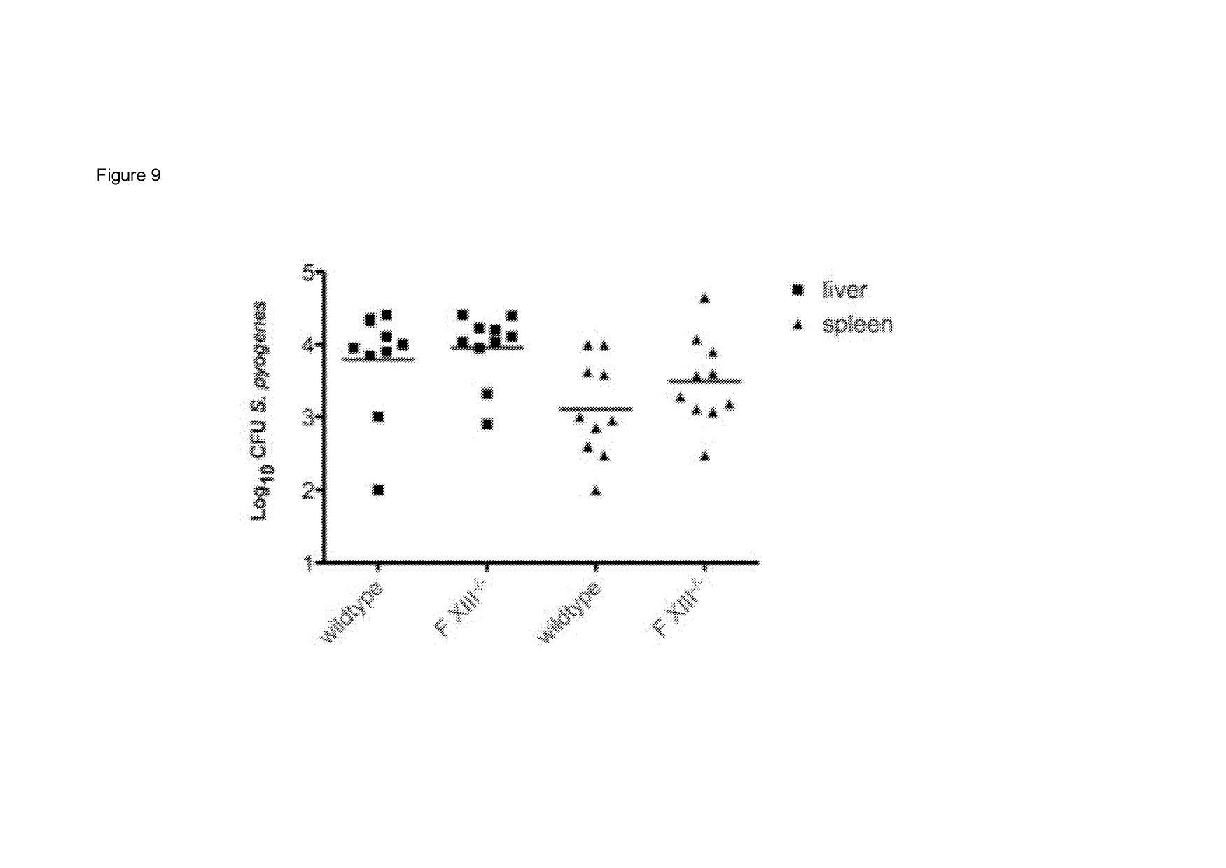

FIG. 9: Bacterial dissemination in infected wildtype and FXIII^{-/-} mice

Wildtype and FXIII^{-/- mice were subcutaneously infected with} S. pyogenes strain KTL3. 24 h after inoculation mice were sacrificed and bacterial loads in blood, liver, and spleen were determined. Data are presented as mean of 10 mice per group and obtained from three independent experiments.

The examples illustrate the invention.

### General Procedures

### Procedure 1: Bacterial Strains and Culture Conditions

The S. pyogenes strain AP1 (40/58) of serotype M1 was originally from the World Health Organization (WHO) Collaborating Center for Reference and Research on Streptococci (Prague, Czech Republic). The S. pyogenes strain KTL3 (M1 serotype) was initially isolated from the blood of a patient with streptococcal bacteremia (Rasmussen et al., 1999). Stock cultures were maintained at −70° C. and were cultured at 37° C. in Todd-Hewitt broth (THB, Gibco; Grand Island, N.Y.). Bacteria were collected in mid-log-phase, washed twice with sterile PBS or Tris, diluted to the required inoculum and the number of viable bacteria was determined by counting colony-forming units (CFU) after diluting and plating in blood agar plates.

### Procedure 2: Human Plasma

Plasma obtained from healthy donors was purchased from Lund University Hospital (Lund, Sweden), plasma kallikrein-(PK)-, thrombin-, F XII-deficient plasma and plasma obtained from patients with FXIII-deficiency (FXIII-deficient plasma) were purchased from George King Bio-Medicals Inc. (Overland Park, Kans.).

### Procedure 3: Substrate Assays

Plasma kallikrein activity on the bacterial surface after exposure to normal, PK-, or FXIII-deficient plasma was measured using the chromogenic substrate S-2302 (Chromogenix, Milan, Italy) as previously described (Oehmcke et al., 2009). For measurement of the thrombin-activity, normal, thrombin-, F XII-, and FXIII-deficient plasma was incubated with 1×10¹⁰ CFU S. pyogenes in 50 mM Tris (pH 7.5) supplemented with 50 μM ZnCl₂, 2 mM CaCl₂, and 1 μM phospholipids (Rossix, MöIndal, Sweden). The tetrapeptide Gly-Pro-Arg-Pro (Bachem, Bubendorf, Switzerland) was added at a final concentration of 1.5 mg/ml to avoid clotting. Samples were incubated for 30 minutes at 37° C., washed twice with Tris and pellets were resuspended in Tris containing 50 μM ZnCl₂ and 1 mM of the chromogenic substrate S-2238 (Chromogenix) and incubated for 30 minutes at 37° C. After centrifugation the absorbance of the supernatants was determined at 405 nm. The FXIII-activity was determined by using a mouse anti-human gold-labeled N-epsilon-gamma-glutamyl-lysine [153-81 D4] antibody (GeneTex, Irvine, Calif.), recognizing the crosslinking site of FXIII. Bacteria were grown overnight as described above and exposed to normal, thrombin-, F XII-, or FXIII-deficient plasma (all diluted 1/100 in sodium citrate) and supplemented with 50 μM ZnCl₂, 2 mM CaCl₂, and 1 μM phospholipids. Samples were incubated in the presence of the gold-labeled antibody for 15 min at 37° C. and analyzed by negative staining electron microscopy.

### Procedure 4: Bacterial Growth in Human Plasma

Bacteria were grown overnight as described above. Human plasma and FXIII-deficient plasma was diluted 1:100 in 12.9 mM sodium citrate and mixed with 500 μl of a solution containing 2.5×10⁵ CFU of S. pyogenes. 0.2 U human thrombin (Sigma, St. Louis, Mo.) was added before incubation at 37° C. After indicated time points 50 μl of the mixture were plated onto blood agar in 10-fold serial dilutions and the number of bacteria was determined by counting colonies after 18 hours of incubation at 37° C. Alternatively, bacteria were also subjected to negative staining electron microscopy.

### Procedure 5: Generation of Plasma Clots

Bacteria were grown overnight as described above. For electron microscopic analysis, 50 μl human or murine plasma (normal and FXIII-deficient) were incubated for 60 seconds at 37° C. in a coagulometer (Amelung, Lemgo, Germany). 2×10⁹ CFU S. pyogenes in 50 μl PBS were added followed by a 60 second incubation. Clotting was then initiated by adding 100 μl thrombin-reagent (Technoclone, Vienna, Austria). Control clots, were generated by adding the thrombin-reagent to plasma in the absence of bacteria. For electron microscopic analysis, clots were fixed in 0.15 M cacodylate buffer (pH 7.2) containing 2.5% glutaraldehyde.

Procedure 6: Crosslinking and Immobilization of Bacteria within the Clot

Fibrinogen purified from human plasma (ICN Biomedicals, Aurora, Ohio) was prepared in a concentration of 300 μg/ml in sodium citrate and incubated with 1 ng/ml recombinant M1 protein in the absence or presence of thrombin-activated human FXIII (Enzyme Research Laboratories, South Bend, Ind.) for 30 min at 37° C. For subsequent visualization by electron microscopy the gold-labeled N-epsilon-gamma-glutamyl-lysine antibody (GeneTex) was given to the reaction mixture. To analyze the bacterial immobilization, clots were generated from normal and FXIII-deficient plasma as described above, washed briefly with PBS and covered with TH-medium. After indicated time points 50 μl of the supernatant were plated onto blood agar in 10-fold serial dilutions and the number of bacteria was determined by counting colonies after 18 hours of incubation at 37° C.

### Procedure 7: Electron Microscopy

For field emission scanning electron microscopy, fixed specimens were washed in cacodylate buffer. Samples were dehydrated with a graded series of ethanol, critical-point dried with CO₂, and sputter coated with gold before examination in a JEOL JSM-350 scanning electron microscope (JEOL Ltd., Tokyo, Japan) operated at 5 kV accelerating voltage and a magnification of 2000. Transmission electron microscopy analysis and immunostaining using the gold-labeled N-epsilon-gamma-glutamyl-lysine antibody was performed as previously described (Bengtsson et al., 2009). For negative staining electron microscopy samples were adsorbed to 400 mesh carbon-coated copper grids for 1 minute, washed briefly with two drops of water, and stained with two drops of 0.75% uranyl formate. The grids were rendered hydrophilic by glow discharge at low pressure in air. Samples were observed in a Jeol 1200 EX transmission electron microscope operated at 60 kV accelerating voltage.

### Procedure 8: Animal Infection Model

CBA/CaOlaHsd wildtype mice were purchased from Harlan (Venray, The Netherlands) and FXIII^{-/- mice were provided by CSL Behring (Marburg, Germany). Mice were housed in a specific pathogen-free animal facility. All animal experiments were approved by the regional ethical committee for animal experimentation, the Malmö/Lund djurforsoksetiska namnd, Lund District Court, Lund, Sweden (permit M}220/08). Before infection, fur was removed from a 2 cm² area on the backs of mice by use of an electric shaver. Mice were subcutaneously infected with 2.5×10⁸ CFU S. pyogenes KTL3 in 100 μl of PBS as previously described (Toppel et al., 2003). After 24 hours of infection mice were sacrificed by CO₂ inhalation. Skin samples were collected by wide marginal excision around the injection site and fixed in 3.7% formaldehyde until histological examination. For plasma analysis, citrated blood was taken from the heart at the time of sacrifice, centrifuged at 5000 rpm for 10 min, and frozen at −80° C. until use. To determine bacterial loads blood and homogenates from liver and spleen were plated in 10-fold serial dilutions onto blood agar. Bacteria colonies were counted after incubation for 18 h at 37° C. In some experiments mice were treated with 200 U/kg body weight of a human FXIII concentrate (Fibrogammin®, CSL Behring) subcutaneously at the site of infection 3 h after bacterial inoculation.

### Procedure 9: Measurement of Coagulation Parameters

Activation of the intrinsic (contact activation) and extrinsic pathway of coagulation was determined by measuring activated partial thromboplastin time (aPTT) and prothrombin time (PT) in plasma of non-infected and infected wildtype and FXIII^{-/- mice, respectively. To measure aPTT,} 50 μl of kaolin (Dapttin TC) (Technoclone, Vienna, Austria) were incubated with 50 μl mouse plasma for 1 min at 37° C. Clotting was initiated by the addition of 50 ml of 25 mM CaCl₂ solution. PT was determined by incubating 50 μl of mouse plasma for 1 min at 37° C. followed by the addition of 50 μl thrombomax reagent (Trinity Biotech, Lemgo, Germany) containing calcium to initiate clotting.

### Procedure 10: Examination of Murine Skin Samples

Mice were subcutaneously infected with 2.5×10⁸ CFU S. pyogenes in 100 μl of PBS, and skin lesions were prepared after 24 h of infection. Tissue samples were fixed in 3.7% formaldehyde, dehydrated in ethanol, embedded in paraffin, and then cut into 3 μm thick sections. After de-paraffination samples were prepared for scanning electron microscopy as described above or stained with haematoxilin and eosin (Histolab, Gothenburg, Sweden) for histological analysis using an Eclipse 80i microscope (Nikon, Tokyo, Japan).

### Procedure 11: Examination of Human Tissue Biopsies

Snap-frozen tissue biopsies collected from the epicenter of infection from two patients with necrotizing fasciitis caused by S. pyogenes of M1T1 serotype were stained and compared with a snap-frozen punch biopsy taken from a healthy volunteer. The Human Subjects Review Committee of the University of Toronto and of Karolinska University Hospital approved the studies, and informed consent was obtained from the patient and the volunteer. The biopsies were cryostat-sectioned to 8 μm, fixed in 2% freshly prepared formaldehyde in PBS. Immunohistochemical staining was done as previously described (Malmström et al., 2009). Immunofluorescent stainings were conducted for M1 protein and N-epsilon-gamma-glutamyl-lysine according to a protocol previously described (Thulin et al., 2006). The following antibodies were used for the immunostainings described above at predetermined optimal dilutions ranging from 1:250-1:10000: anti N-epsilon-gamma-glutamyl-lysine (GeneTex), anti-factor XIIIa (Acris, Herford, Germany), a polyclonal rabbit antiserum specific for the Lancefield group A carbohydrate (Difco) as well as a polyclonal rabbit antiserum against M1. The immunohistochemical stainings were evaluated in a RXM Leica microscope with a 25×/0.55 NA oil objective lens (Leica, Wetzlar, Germany), while the immunofluorescent stainings were evaluated and visualized using a Leica confocal scanner TCS SP II coupled to a Leica DMR microscope.

### Procedure 12: Statistical Analysis

Data were analyzed by using Excel 2007 (Microsoft Office, Microsoft, Redmont, Wash.) or GraphPad Prism 5 (GraphPad Software, San Diego, Calif.). The significance between the values of an experimental group was determined by use of a variance analysis (t test). Significance levels were set at P<0.05.

### Example 1

### Contact Activation at the Surface of S. pyogenes Leads to an Induction of FXIII

Previous work has shown that the presence of S. pyogenes in plasma leads to an assembly and activation of the contact system at the bacterial surface (Herwald et al., 2003). These experiments were performed in the absence of calcium and phospholipids, which are important co-factors in hemostasis and required for an activation of coagulation factors up-stream of the contact system (for a review see (Hoffman and Monroe, 2001)). The inventors therefore wondered whether calcium and phospholipid reconstitution triggers an induction of the remaining clotting cascade at the bacterial surface. To confirm the previous findings the inventors first measured plasma kallikrein activity on AP1 bacteria upon incubation with normal zincified human plasma. Plasma kallikrein-deficient and FXIII-deficient plasma served as controls in these experiments. As depicted in FIG. 1A, substrate hydrolysis was monitored when bacteria were incubated with normal and FXIII-deficient plasma, but not when plasma was deficient of plasma kallikrein. These findings are in line with the previous reports and it was therefore studied next whether bacteria-induced contact activation leads to an induction of the entire coagulation cascade by measuring thrombin activity, the activator of FXIII. To this end, normal plasma was reconstituted with zinc, calcium, and phospholipids. Samples were also supplemented with a tetrapeptide (Gly-Pro-Arg-Pro) to avoid polymerization of thrombin-generated fibrin monomers and subsequent a coagel formation (for detailed information see Experimental Procedures). When this reaction mixture was added to normal plasma and incubated with AP1 bacteria, an increase of thrombin activity at the bacterial surface was monitored (FIG. 1B). Similar results were also obtained with FXIII-deficient, but not with FXII-deficient or thrombin-deficient plasma, implying that activation of the contact system at the bacterial surface is required to trigger activation of the remaining clotting factors (FIG. 1B). FXIII is one of thrombin's substrates and it was therefore tested whether bacteria-induced thrombin activation triggers a conversion of FXIII into its active form. To this end, the inventors employed an antibody directed against N-epsilon-gamma-glutamyl-lysine which specifically recognizes amino acids that are covalently crosslinked by the action of FXIII (el Alaoui et al., 1991). As Gly-Pro-Arg-Pro exerted a mild bacteriostatic effect in the experiments, it was decided not to use this peptide as anti-coagulant. Instead, plasma was diluted to a concentration (1/100) in which the fibrin concentration was too low to cause its polymerization when activated by thrombin. Bacteria were incubated with diluted normal, thrombin-, F XII-, and FXIII-deficient plasma in the presence of the gold-labeled antibody, zinc, calcium, and phospholipids. Samples were then analyzed by negative staining electron microscopy. FIG. 1C shows antibody binding to the surface of S. pyogenes bacteria treated with normal diluted plasma, while only background signals were detected, when bacteria were incubated with F XII- or FXIII-deficient plasma (FIG. 1C). Similar results were obtained with thrombin-deficient plasma (data not shown). Taken together these results suggest that contact activation at the bacterial surface when exposed to plasma, can evoke an induction of the entire coagulation cascade and eventually enables FXIII to act on S. pyogenes surface proteins.

### Example 2

### Streptococci are Killed in Thrombin-Activated but not in Non-Activated Plasma

In the next series of experiments the inventors wished to study the fate of crosslinked bacteria in activated, but non-clotted, normal and FXIII-deficient plasma. FIG. 2A shows that bacterial growth is significantly impaired in thrombin-activated normal and FXIII-deficient plasma. This effect was time dependent and was not seen when plasma was left non-activated. To study whether the activation of plasma was combined with an induction of antimicrobial activity, plasma-treated bacteria were subjected to negative staining electron microscopy. FIG. 2B (left panel) depicts intact bacteria that were incubated with non-activated normal plasma and similar findings were observed when bacteria were incubated with non-activated FXIII-deficient plasma (data not shown). Once activated with thrombin, however, incubation with normal plasma (FIG. 2B, middle panel) and FXIII-deficient plasma (FIG. 2B, right panel) caused multiple disruptions of the bacterial cell wall and triggered an efflux of cytosolic content which is a sign of bacterial killing (Malmström et al., 2009). Notably, incubation with thrombin in the absence of plasma neither impaired bacterial growth nor did it cause cytosolic leakage (data not shown).

To test whether bacterial killing also occurs within a formed clot, AP1 bacteria and undiluted plasma were mixed followed by activation with thrombin. Formed coagels were incubated for 1 h, thin-sectioned and analyzed by transmission electron microscopy. FIG. 2C displays that most bacteria in clots generated from normal and FXIII-deficient plasma (lower panel) are devoid of cytosolic content, suggesting a substantial disruption of the cell membrane and bacterial killing. By contrast only a few dead bacteria were seen when clots were thin-sectioned directly after the addition of thrombin (upper panel). Together these data demonstrate that the coagulation cascade bears antimicrobial activity that is exposed upon its activation, but independent of FXIII.

### Example 3

### Bacterial Entrapment within a Plasma Clot is FXIII-Dependent

Human FXIII was recently shown in vitro to crosslink and immobilize bacteria of species Staphylococcus aureus and Escherichia coli inside a plasma clot (Wang et al., 2010). To test whether this also applies to S. pyogenes, AP1 bacteria were incubated with normal and FXIII-deficient plasma and thrombin-activated clots were analyzed by scanning electron microscopy. FIGS. 3A and 3B show clots formed from normal and FXIII-deficient plasma in the absence of bacteria. The micrographs reveal that both types of clots share a similar morphology, although clots generated from FXIII-deficient plasma appear to be less dense. However, dramatic changes were observed when clots were formed in the presence of AP1 bacteria. While massive loads of bacteria were entrapped in clots derived from normal plasma (FIG. 3C), only a few bacteria were found attached to clots when FXIII-deficient plasma was used (FIG. 3D). Also, fibrin network formation was reduced when bacteria were incubated with normal plasma, which was not seen when FXIII-deficient plasma was employed (FIGS. 3C and 3D). At higher resolution it is noticeable that fibrin fibers and bacteria are in close proximity in the clots generated from normal plasma and it even appears that fibers originate from the bacterial surface (FIG. 3E). By contrast, bacteria are loosely assembled in clots from FXIII-deficient plasma and no direct interaction with fibrin fibers is detectable (FIG. 3F). To confirm these findings, clots from normal plasma were thin-sectioned and subjected to transmission electron microscopy, which allows an analysis at higher resolutions. FIG. 3G-I depicts thin-sectioned AP1 bacteria before (FIG. 3G) and directly after incubation with normal plasma and subsequent thrombin-activation (FIG. 3H). Within the clot, bacteria are strung along fibrin fibers and it appears that they have multiple interactions sites. Additional immunostaining with the gold-labeled antibody against N-epsilon-gamma-glutamyl-lysine was used to study the mode of interaction between bacteria and fibrin fibers. Numerous crosslinking events within fibrin fibers were detected. The electron microscopic analysis also revealed that fibrin fibers are avidly crosslinked to the surface of AP1 bacteria (FIG. 3I). Crosslinking activity was not recorded when bacteria were incubated with FXIII-deficient plasma (data not shown).

Most streptococcal serotypes have a high affinity for fibrinogen and the M1 protein has been reported to be the most important fibrinogen receptor of the AP1 strain (Åkesson et al., 1994). The respective bindings sites were mapped to amino-term inal region of M1 protein and fragment D, which is part of the terminal globular domain of fibrinogen (Åkesson et al., 1994). Negative staining electron microscopy was employed to study the interaction of M1 protein and fibrinogen at the molecular level. The results demonstrate that one terminal region of the streptococcal surface protein is in complex with a globular domain of fibrinogen (FIG. 4A, upper panel), which is in good agreement with the mapping study. The nature of this complex was not altered when activated FXIII was co-incubated with the two proteins (FIG. 4A, middle panel). Indeed, additional immuno-detection with the gold-labeled antibody against N-epsilon-gamma-glutamyl-lysine revealed that the interaction site is covalently crosslinked by FXIII (FIG. 4A, lower panel). As M proteins are the most abundant surface proteins of streptococci it seems plausible that M1 protein of AP1 bacteria is one of the major interaction partners that is covalently attached to fibrin fibers by the action of FXIII. However, it cannot be excluded that also other streptococcal surface proteins are targeted by FXIII.

Whether crosslinking of bacteria by FXIII has a pathophysiologic function inside the clot, was studied by measuring the escape of AP1 bacteria from clots generated from normal and FXIII-deficient plasma. To this end Streptococci were mixed with undiluted normal or FXIII-deficient plasma and clotting was induced by the addition of thrombin. Clots were then briefly washed with PBS and covered with growth medium. After different time points samples were collected form the supernatant and their bacterial load was determined. As seen in FIG. 4B, FXIII-induced crosslinking significantly reduced the release of bacteria from the clot suggesting their immobilization and killing within the clot. Taken together, the results show that S. pyogenes bacteria are covalently weaved into a fibrin network by the action of FXIII and this prevents their dissemination from the clot.

### Example 4

### S. pyogenes Infected FXIII^{-/-} Mice have More Signs of Inflammation than Wildtype Animals

The in vitro data suggest that coagulation is part of the early innate immune response, which in a concert action triggers an immobilization and killing of S. pyogenes inside a clot. The inventors therefore hypothesized that prevention of bacterial dissemination and their clearance may dampen the inflammatory response at the site of infection. To test this, it was taken advantage of a skin infection model that was established with another M1 serotype, KTL3 respectively (Toppel et al., 2003). Challenge with the KTL3 strain normally causes local infections that eventually disseminate from the infection focus and lead to systemic infections (Toppel et al., 2003). By employing scanning electron microscopic analysis, it was found that incubation of the KTL3 strain with thrombin-activated human normal or FXIII-deficient plasma in vitro generates clots with a morphology similar to those generated with AP1 bacteria (data not shown). Similar results were also obtained when murine plasma (normal and FXIII-deficient) was incubated with KTL3 bacteria (FIG. 8).

To study the inflammatory response to a local infection with S. pyogenes, wildtype and FXIII^{-/-} mice were subcutaneously infected with the KTL3 strain. 24 h after infection, mice were sacrificed and the skin from the local focus of infection was surgically removed and stained with hematoxylin and eosin for histopathological analysis. While the microscopic examination of skin biopsies from non-infected wildtype and FXIII^{-/-} mice revealed no signs of inflammation (FIG. 5A+B), edema formation, neutrophils invasion, and tissue damage was seen in biopsies from infected wildtype animals (FIG. 5C). Notably, these lesions were by far more severe when biopsies from infected FXIII^{-/-} mice were microscopically analyzed (FIG. 5D).

Further electron microscopic examination of the tissue biopsies from wildtype and FXII^{-/-} mice showed severe bleeding all over the infected site (data not shown). However, bacteria were found entrapped and clustered within the fibrin meshwork of infected wildtype mice (FIG. 5E), whereas they were scattered all over the clot when skin biopsies from infected FXIII^{-/-} mice were analyzed (FIG. 5F). Additional statistical analysis revealed approximately 8 bacterial clusters pro 100 μm² in the fibrin network of wildtype animals, while streptococci were seen mostly as single bacteria or small chains at a density of 41 bacteria/chains pro 100 μm². At higher magnification it appears that bacteria are an integral part of the fibrin network from infected wildtype mice (FIG. 5E, insert). This was not observed in biopsies from FXIII mice where streptococci were found associated with but not as a constituent of the network (FIG. 5F, insert). Whether the immobilization of bacteria influenced their dissemination was investigated by measuring clotting times of the intrinsic pathway of coagulation (activated partial thromboplastin time or aPTT), which, if increased, is a sign of a systemic response to the infection (Oehmcke et al., 2009). To this end, mice were infected for 24 h. Thereafter plasma samples were recovered and clotting times of the intrinsic pathway of coagulation determined. FIG. 5G shows that the aPTTs of plasma samples from infected wildtype mice were moderately but significantly increased, while clotting times were skyrocketed in plasma samples from FXIII^{-/-} mice. The prothrombin time (PT) remained unaltered after 24 h of infection in both groups of mice (data not shown). The analysis of bacterial load in liver and spleen showed slightly increased levels of bacteria especially in the spleens of FXIII^{-/-} mice, but when compared with wildtype animals the differences were not significant (FIG. 9). Together these results demonstrate that the lack of FXIII leads to an increased inflammatory response at the infectious site combined with an induction of systemic reactions.

### Example 5

### FXIII Crosslinking in Patients with Necrotizing Fasciitis Caused by S. pyogenes

To test whether the results obtained from the animal studies also apply to the clinical situation, biopsies from patients with necrotizing fasciitis caused by S. pyogenes were analyzed by immunohistological and electron microscopic means. FIG. 6 depicts massive tissue necrosis at the site of infection and subsequent immunodetection showed positive staining for the M1 protein and FXIII at these sites. This suggests an influx of plasma to the infected focus and indeed an increased crosslinking activity at these sites was recorded (FIG. 6, upper lane). As controls, biopsies from healthy persons were used, but no immunostaining was recorded when the biopsies were subjected to the same experimental protocol (FIG. 6, lower lane). Tissue sections were further analyzed by confocal immuno-fluorescence microscopy using antibodies against the M1 protein and N-epsilon-gamma-glutamyl-lysine. FIG. 7A shows co-localization of the two antibodies suggesting bacterial crosslinking at the infected site. When the biopsies were analyzed by scanning electron microscopy, massive bleeding at the infected site was recorded (data not shown) and bacteria were found clustered and entrapped inside the fibrin network (FIG. 7B). Specimens were also thin-sectioned and studied by immuno transmission electron microscopy using the gold-labeled antibody against N-epsilon-gamma-glutamyl-lysine. FIG. 7C depicts immunostaining at the bacterial surface in regions that are in contact with fibrin fibers. The micrographs also reveal that a significant portion of the entrapped bacteria were not viable as shown in FIG. 7D. These findings are in line with the in vitro and in vivo experiments and they illustrate that immobilization of bacteria and generation of antimicrobial activity is seen in clots from patients with severe and invasive infections with S. pyogenes.

### Example 6

### Local Treatment with FXIII Dampens Systemic Bacterial Spreading in Infected Mice

To test whether treatment with FXIII is able to prevent bacterial spreading in an animal model of infection, wildtype mice were subcutaneously infected with S. pyogenes. Three hours after challenge, half of the mice were treated with Fibrogammin®, a human plasma FXIII concentrate, which was injected into the site of infection. A dose of 200 international units (IU) per kg body weight was chosen, which is approximately 10 times as much as the normal plasma levels and this has been shown to be well tolerated in mice (Lauer et al., 2002). Mice infected with S. pyogenes but without fibrogammin-treatment served as controls. 24 h after infection animals were sacrificed and bacterial loads in blood, liver, and spleen were determined. As shown in FIG. 7E in all three cases significantly lower amounts of bacteria were found in the fibrogammin-treated mice, suggesting FXIII dampens systemic dissemination of S. pyogenes in the infected animals. Taken together, the results presented in connection with this invention support the concept of an early defense system against bacterial infections involving a FXIII-mediated immobilization of bacteria inside the clot combined with an induction of plasma-derived antimicrobial activity and subsequent bacterial killing. The data suggest that these two mechanisms work in a concert action and this may diminish bacterial dissemination and down-regulate the inflammatory response.

### REFERENCES

Åkesson, P., Schmidt, K. H., Cooney, J., and Björck, L. (1994). M1 protein and protein H: IgGFc- and albumin-binding streptococcal surface proteins encoded by adjacent genes. Biochem J 300, 877-886.
Bidla, G., Hauling, T., Dushay, M. S., and Theopold, U. (2009). Activation of insect phenoloxidase after injury: Endogenous versus foreign elicitors. J Innate Immun 1, 301-308.
Carapetis, J. R., Steer, A. C., Mulholland, E. K., and Weber, M. (2005). The global burden of group A streptococcal diseases. Lancet Infect Dis 5, 685-694.
Cunningham, M. W. (2000). Pathogenesis of group A streptococcal infections. Clin Microbiol Rev 13, 470-511.
Davidson, C. J., Tuddenham, E. G., and McVey, J. H. (2003). 450 million years of hemostasis. J Thromb Haemost 1, 1487-1494.
Delvaeye, M., and Conway, E. M. (2009). Coagulation and innate immune responses: can we view them separately? Blood 114, 2367-2374.
Dharmani, P., Srivastava, V., Kissoon-Singh, V., and Chadee, K. (2009). Role of intestinal mucins in innate host defense mechanisms against pathogens. J Innate Immun 1, 123-135.
Eisen, D. P. (2010). Mannose-binding lectin deficiency and respiratory tract infection. J Innate Immun 2, 114-122.
el Alaoui, S., Legastelois, S., Roch, A. M., Chantepie, J., and Quash, G. (1991). Transglutaminase activity and N epsilon (gamma glutamyl) lysine isopeptide levels during cell growth: an enzymic and immunological study. Int J Cancer 48, 221-226.
Endo, Y., Nakazawa, N., Iwaki, D., Takahashi, M., Matsushita, M., and Fujita, T. (2009). Interactions of ficolin and mannose-binding lectin with fibrinogen/fibrin augment the lectin complement pathway. J Innate Immun 2, 33-42.
Frick, I. M., Åkesson, P., Herwald, H., Mörgelin, M., Malmsten, M., Nagler, D. K., and Björck, L. (2006). The contact system—a novel branch of innate immunity generating antibacterial peptides. Embo J 25, 5569-5578.
Frick, I. M., Björck, L., and Herwald, H. (2007). The dual role of the contact system in bacterial infectious disease. Thromb Haemost 98, 497-502.
Gershom, E. S., Sutherland, M. R., Lollar, P., and Pryzdial, E. L. (2010). Involvement of the contact phase and intrinsic pathway in herpes simplex virus-initiated plasma coagulation. J Thromb Haemost 8, 1037-1043.
Herwald, H., Mörgelin, M., Dahlbäck, B., and Björck, L. (2003). Interactions between surface proteins of Streptococcus pyogenes and coagulation factors modulate clotting of human plasma. J Thromb Haemost 1, 284-291.
Hoffman, M., and Monroe, D. M., 3rd (2001). A cell-based model of hemostasis. Thromb Haemost 85, 958-965.
Krem, M. M., and Di Cera, E. (2001). Molecular markers of serine protease evolution. EMBO J. 20, 3036-3045.
Krem, M. M., and Di Cera, E. (2002). Evolution of enzyme cascades from embryonic development to blood coagulation. Trends Biochem Sci 27, 67-74.
Lauer, P., Metzner, H. J., Zettlmeissl, G., Li, M., Smith, A. G., Lathe, R., and Dickneite, G. (2002). Targeted inactivation of the mouse locus encoding coagulation factor XIII-A: hemostatic abnormalities in mutant mice and characterization of the coagulation deficit. Thromb Haemost 88, 967-974.
Leeb-Lundberg, L. M. F., Marceau, F., Müller-Esterl, W., Pettibone, D. J., and Zuraw, B. L. (2005). International union of pharmacology. XLV. Classification of the kinin receptor family: from molecular mechanisms to pathophysiological consequences. Pharmacol Rev 57, 27-77.
Malmström, E., Mörgelin, M., Malmsten, M., Johansson, L., Norrby-Teglund, A., Shannon, O., Schmidtchen, A., Meijers, J. C., and Herwald, H. (2009). Protein C inhibitor—a novel antimicrobial agent. PLoS Pathog 5, e1000698.
Nonaka, M., and Kimura, A. (2006). Genomic view of the evolution of the complement system. Immunogenetics 58, 701-713.
Nordahl, E. A., Rydengård, V., Mörgelin, M., and Schmidtchen, A. (2005). Domain 5 of high molecular weight kininogen is antibacterial. J Biol Chem 280, 34832-34839.
Oehmcke, S., Shannon, O., von Köckritz-Blickwede, M., Mörgelin, M., Linder, A.,
Olin, A. I., Björck, L., and Herwald, H. (2009). Treatment of invasive streptococcal infection with a peptide derived from human high-molecular weight kininogen. Blood 114, 444-451.
Opal, S. M., and Esmon, C. T. (2003). Bench-to-bedside review: Functional relationships between coagulation and the innate immune response and their respective roles in the pathogenesis of sepsis. Crit. Care 7, 23-38.
Page, M. J., and Di Cera, E. (2008). Serine peptidases: classification, structure and function. Cell Mol Life Sci 65, 1220-1236.
Papareddy, P., Rydengard, V., Pasupuleti, M., Walse, B., Mörgelin, M., Chalupka, A., Malmsten, M., and Schmidtchen, A. (2010). Proteolysis of human thrombin generates novel host defense peptides. PLoS Pathog 6, e1000857.
Rapala-Kozik, M., Karkowska, J., Jacher, A., Golda, A., Barbasz, A., Guevara-Lora, I., and Kozik, A. (2008). Kininogen adsorption to the cell surface of Candida spp. International immunopharmacology 8, 237-241.
Toppel, A. W., Rasmussen, M., Rohde, M., Medina, E., and Chhatwal, G. S. (2003). Contribution of protein G-related alpha2-macroglobulin-binding protein to bacterial virulence in a mouse skin model of group A streptococcal infection. J Infect Dis 187, 1694-1703.
Wang, Z., Wilhelmsson, C., Hyr\check{s}l P., Loof, T. G., Dobe\check{s}, P., Klupp, M., Loseva, O., Mörgelin, M., Iklé, J., Cripps, R. M., et al. (2010). Pathogen Entrapment by Transglutaminase—a Conserved Early Innate Immune Mechanism. PLoS Pathog, 6:e1000763.

1.-14. (canceled)

15. A method of treatment and/or prevention of an infection by a microorganism and/or the symptoms associated with said infection comprising:
administering to a patient in need thereof a pharmaceutically effective amount of a blood coagulation factor XIII (FXIII).

16. The method according to claim 15, wherein treatment and/or prevention comprises
(i) administering FXIII to a patient so that the FXIII concentration in the blood plasma of that patient is increased above the FXIII concentration in the blood plasma of a healthy individual, and/or
(ii) administering FXIII to a patient so that an initial concentration of FXIII in the patient's blood plasma is up to 10 fold at its normal level and/or
(iii) administering FXIII to a patient who does not suffer from a congenital or acquired FXIII deficiency.

17. The method of claim 15, wherein said FXIII is administered to said patient as part of a pharmaceutical composition.

18. The method according to claim 17, wherein FXIII is administered to a patient systemically or topically to an infected area.

19. The method according to claim 17, wherein FXIII is administered to a patient at a dose of 5 to 1000 international units (IU) per kg body weight.

20. The method according to claim 17, wherein said administering results in dampening systemic dissemination, immobilization and/or killing of the microorganism in the body of a patient.

21. The method according to claim 15, wherein the microorganism is capable of supporting or enhancing fibrinolysis, is capable of activating plasminogen and/or has a plasminogen activating protein selected from the group consisting of streptokinase, staphylokinase, protein Pla, fibrinolytic enzymes, compounds that activate fibrinolysis or other bacterial proteins.

22. The method according to claim 15, wherein the microorganism is capable of supporting or enhancing fibrinolysis by carrying at least one surface and/or cell wall protein capable of lowering the plasma concentration of at least one inhibitor of plasminogen activation.

23. The method according to claim 15, wherein the microorganism is selected from the group consisting of bacteria, yeasts, viruses and multicellular parasites.

24. The method according to claim 15, wherein the infection is of one or more tissues selected from the group consisting of skin, respiratory system, throat, lung, spleen, liver, kidney, cardiovascular system, heart, central nervous system, digestive system, genitourinary system, muscles and soft tissues.

25. The method according to claim 15, wherein the symptoms are selected from the group consisting of inflammation, headaches, fever, diarrhea, pain, loss of consciousness and a combination thereof.

26. The method according to claim 18, wherein the FXIII is administered topically to an infected area.

27. The method according to claim 19, wherein FXIII is administered to a patient at a dose of 10 to 200 IU per kg body weight.

28. The method according to claim 22, wherein said protein is selected from the group consisting of protein GRAB (Streptococcus pyogenes), aureolysin (Staphylococcus aureus), secreted neutral metalloproteases of Bacillus anthracis, and secreted proteases of Peptostreptococcus micros.

29. The method according to claim 23, wherein the bacteria has a solid cell wall and/or is Gram-positive.

30. The method according to claim 23, wherein the bacteria is an aerobe or facultative anaerobe coccobacilli.

31. The method according to claim 23, wherein the bacteria is a Streptococcaceae.

32. The method according to claim 23, wherein the bacteria is a hemolytic Streptococci.

33. The method according to claim 23, wherein the bacteria is Streptococcus pyogenes.

34. The method according to claim 15, wherein the infection is a systemic infection of the body of the patient.

35. The method of claim 15, wherein said FXIII is administered as a concentrate.

36. The method of claim 15, wherein said FXIII has been isolated from human blood plasma or is provided as a recombinant protein.
